(19) United States
(12) Patent Application Publication
(10) Pub. No.: US 2025/0360166 A1
(43) Pub. Date: Nov. 27, 2025

(54) Cisplatin particles and uses thereof

(71) Applicant: CRITITECH, INC., Lawrence, KS (US)

(72) Inventors: Jacob SITTENAUER, Lawrence, KS (US); Aranza Barreda ABARCA, Lawrence, KS (US); Joseph FARTHING, Lawrence, KS (US); Mark WILLIAMS, Lawrence, KS (US); Michael BALTEZOR, Lawrence, KS (US)

(21) Appl. No.: 19/293,697

(22) Filed: Aug. 7, 2025

Related U.S. Application Data
(63) Continuation of application No. 18/556,705, filed on Oct. 23, 2023.
(60) Provisional application No. 63/179,855, filed on Apr. 26, 2021.

(51) Int. Cl. A61K 33/243; A61K 9/12; A61K 9/16; A61P 31/00

(52) U.S. Cl. CPC A61K 33/243; A61K 9/12; A61K 9/1629; A61P 31/00

(57) ABSTRACT

Compositions of particles having at least 95% by weight of cisplatin and a specific surface area (SSA) of at least 3.5 m²/g. methods for their use. and methods for their production are provided.

### BACKGROUND

Dissolution rate is a key parameter in determining the rate and extent of drug absorption and bioavailability. Poor aqueous solubility and poor in vivo dissolution are limiting factors for in vivo bioavailability of many drugs. Thus, in vitro dissolution rates are recognized as an important element in drug development, and methods and compositions for increasing the dissolution rates of poorly soluble drugs are needed.

### SUMMARY

In one aspect, the disclosure provides compositions, comprising particles comprising at least 95% by weight of cisplatin, wherein the particles have a specific surface area (SSA) of at least 3.5 m²/g. In various embodiments, the particles have a SSA of at least 4 m²/g or at least 10 m²/g. In other embodiments, the particles have a SSA of between 3.5 m²/g and about 50 m²/g. In one embodiment, the particles have a mean particle size by volume distribution (Dv50) of between about 1.0 micron to about 12 microns in diameter. In another embodiment, wherein the particles have a mean bulk density between about 0.020 g/cm³ and about 0.8 g/cm³. In one embodiment, the composition comprises a suspension. In one embodiment, the suspension is aerosolized, and the mass median aerodynamic diameter (MMAD) of aerosol droplets of the suspension is between about 0.5 μm to about 6 μm diameter. In other embodiments, the composition is a dry powder composition, wherein (a) the dry powder composition does not comprise a carrier or any excipients, wherein the dry powder composition is aerosolized, and the MMAD of the aerosolized dry powder composition may be any suitable diameter for use, such as between about 0.5 μm to about 6 μm in diameter, or (b) the composition is a dry powder composition, wherein the dry powder composition comprises a pharmaceutically acceptable dry powder carrier comprising one or more dry powder excipients, and wherein the dry powder composition is aerosolized, and the MMAD of the aerosolized dry powder composition may be any suitable diameter for use, such as between about 0.5 μm to about 6 μm in diameter.

In another aspect, the disclosure provides methods for treating a tumor, comprising administering to a subject with a tumor an amount effective to treat the tumor of the composition of any embodiment or combination of embodiments herein.

In a further aspect, the disclosure provides methods for making compound particles, comprising:
(a) introducing (i) a solution comprising at least one solvent including but not limited to DMF (dimethylformamide), DMSO (dimethyl sulfoxide), acetone, or combinations thereof, or combinations thereof, and at least one solute comprising cisplatin into a nozzle inlet, and (ii) a compressed fluid into an inlet of a vessel defining a pressurizable chamber;
(b) passing the solution out of a nozzle orifice and into the pressurizable chamber to produce an output stream of atomized droplets, wherein the nozzle orifice is located between 2 mm and 20 mm from a sonic energy source located within the output stream, wherein the sonic energy source produces sonic energy with an amplitude between 10% and 100% during the passing, and wherein the nozzle orifice has a diameter of between 20 μm and 125 μm;
(c) contacting the atomized droplets with the compressed fluid, to cause depletion of the solvent from the atomized droplets, to produce cisplatin particles comprising at least 95% cisplatin, wherein the cisplatin particles have a specific surface area (SSA) of at 3.5 m²/g and have a mean particle size of between about 0.7 μm and about 8 μm,
wherein steps (a), (b), and (c) are carried out under supercritical temperature and pressure for the compressed fluid.

### DESCRIPTION OF THE FIGURES

FIG. 1(A-B). Scanning Electron Microscopy Micrographs (A) Raw material cisplatin 1000X, (B) Raw material cisplatin 5000X.

FIG. 2(A-B). Scanning Electron Microscopy Micrographs of cisplatin SC1 processed using DMF as solvent at (A) 2000X magnification, and (B) 10,000X magnification.

FIG. 3(A-B). Scanning Electron Microscopy Micrographs cisplatin SC2 processed using DMSO as solvent at (A) 2000X magnification, and (B) 10,000X magnification.

FIG. 4(A-B). Scanning Electron Microscopy Micrographs of cisplatin SC3processed using 3:2 DMSO: Acetone at (A) 2000X magnification, and (B) 10,000X magnification. FIG. 5(A-B). Scanning Electron Microscopy Micrographs of cisplatin SC4 processed using high pressure at (A) 2000X magnification, and (B) 10,000X magnification.

FIG. 6(A-B). Scanning Electron Microscopy Micrographs of cisplatin SC5 processed using low pressure at (A) 2000X magnification, and (B) 10,000X magnification.

FIG. 7(A-B). Scanning Electron Microscopy Micrographs of cisplatin SC6 processed using low temperature at (A) 2000X magnification, and (B) 10,000X magnification.

FIG. 8(A-B). Scanning Electron Microscopy Micrographs of cisplatin SC7 processed using high temperature at (A) 2000X magnification, and (B) 10,000X magnification.

FIG. 9(A-B). Scanning Electron Microscopy Micrographs of cisplatin SC8 processed using high scCO₂ flow at (A) 2000X magnification, and (B) 10,000X magnification.

FIG. 10(A-B). Scanning Electron Microscopy Micrographs of cisplatin SC9 processed using low scCO₂ flow at (A) 2000X magnification, and (B) 10,000X magnification.

FIG. 11(A-B). Scanning Electron Microscopy Micrographs of cisplatin SC10 processed using high sonication at (A) 2000X magnification, and (B) 10,000X magnification. FIG. 12(A-B). Scanning Electron Microscopy Micrographs of cisplatin SC11processed using low sonication at (A) 2000X magnification, and (B) 10,000X magnification. FIG. 13(A-B). Scanning Electron Microscopy Micrographs of cisplatin SC12 processed using no sonication at (A) 2000X magnification, and (B) 10,000X magnification. FIG. 14(A-B). Scanning Electron Microscopy Micrographs of cisplatin SC13 processed using low temperature and low sonication at (A) 2500X magnification, and (B) 10,000X magnification.

FIG. 15(A-B) Powder X-ray Diffraction Patterns for (A) cisplatin runs SC1-SC6, and (B) cisplatin runs SC7-SC13, compared to the cisplatin raw material.

FIG. 16. Graph showing treatment effect on mean tumor volume as a function of time.

FIG. 17. Graph showing effect of IT cisplatin treatment on mean tumor volume as a function of time in individual test subjects.

FIG. 18. Graph showing effect of IT SCP-cisplatin low dose treatment on mean tumor volume as a function of time in individual test subjects.

FIG. 19. Graph showing effect of IT SCP-cisplatin high dose treatment on mean tumor volume as a function of time in individual test subjects.

### DETAILED DESCRIPTION

All references cited are herein incorporated by reference in their entirety. As used herein, the singular forms “a”, “an” and “the” include plural referents unless the context clearly dictates otherwise. All embodiments of any aspect of the disclosure can be used in combination, unless the context clearly dictates otherwise.

As used herein, “about” means +/−5% of the recited value.

In one aspect, the disclosure provides compositions, comprising particles comprising at least 95% by weight of cisplatin, wherein the particles have a specific surface area (SSA) of at least 3.5 m²/g.

As used herein, “cisplatin” includes any ionization state of cisplatin, including base, acid, and neutral states.

### Structure of Cisplatin

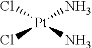

Cisplatin molecular formula: Pt(NH₃)₂Cl₂

The “cisplatin particles” refers to particles of cisplatin that do not include an added excipient. Cisplatin particles are different than “particles containing cisplatin”, which are particles that contain cisplatin and at least one added excipient. Cisplatin particles of the disclosure exclude a polymeric, wax or protein excipient and are not embedded, contained, enclosed or encapsulated within a solid excipient. Cisplatin particles of the disclosure may, however, contain impurities and byproducts typically found during preparation of cisplatin. Even so, cisplatin particles comprise at least 95%, at least 96%, at least 97%, at least 98%, at least 99% or 100% cisplatin, meaning the cisplatin particles consist of or consist essentially of substantially pure cisplatin.

As used herein, the “specific surface area” is the total surface area of the cisplatin particle per unit of cisplatin mass as measured by the Brunauer-Emmett-Teller (“BET”) isotherm (i.e.: the BET SSA). As will be understood by those of skill in the art, the SSA is determined on a per gram basis and takes into account both agglomerated and non-agglomerated cisplatin particles in the composition. The BET specific surface area test procedure is a compendial method included in both the United States Pharmaceopeia and the European Pharmaceopeia. The cisplatin particles have a specific surface area (SSA) of at least 3.5 m²/g. In various further embodiments, the cisplatin particles have a SSA of at least 4 m²/g, 5 m²/g, 6 m²/g, 7 m²/g, 8 m²/g, 9 m²/g, 10 m²/g, 11 m²/g, 12 m²/g, 13 m²/g, 14 m²/g, 15 m²/g, 16 m²/g, 17 m²/g, 18 m²/g, 19 m²/g,, 20 m²/g,, 21 m²/g, 22 m²/g, 23 m²/g, or 24 m²/g.

In further embodiments, the cisplatin particles have a SSA of between 3.5 m²/g and about 50 m²/g, about 4 m²/g and about 50 m²/g, about 5 m²/g and about 50 m²/g, between about 6 m²/g and about 50 m²/g, between about 7 m²/g and about 50 m²/g, between about 8 m²/g and about 50 m²/g, between about 7 m²/g and about 50 m²/g, between about 9 m²/g and about 50 m²/g, between about 10 m²/g and about 50 m²/g, between about 11 m²/g and about 50 m²/g, between about 12 m²/g and about 50 m²/g, between about 13 m²/g and about 50 m²/g, between about 14 m²/g and about 50 m²/g, between about 15 m²/g and about 50 m²/g, between about 16 m²/g and about 50 m²/g, between about 17 m²/g and about 50 m²/g, between about 18 m²/g and about 50 m²/g, between about 19 m²/g and about 50 m²/g, between about 20 m²/g and about 50 m²/g, between about 21 m²/g and about 50 m²/g, between about 22 m²/g and about 50 m²/g, between about 23 m²/g and about 50 m²/g, between about 24 m²/g and about 50 m²/g, between 3.5 m²/g and about 45 m²/g, about 4 m²/g and about 45 m²/g, about 5 m²/g and about 45 m²/g, between about 6 m²/g and about 45 m²/g, between about 7 m²/g and about 45 m²/g, between about 8 m²/g and about 45 m²/g, between about 7 m²/g and about 45 m²/g, between about 9 m²/g and about 45 m²/g, between about 10 m²/g and about 45 m²/g, between about 11 m²/g and about 45 m²/g, between about 12 m²/g and about 45 m²/g, between about 13 m²/g and about 45 m²/g, between about 14 m²/g and about 45 m²/g, between about 15 m²/g and about 45 m²/g, between about 16 m²/g and about 45 m²/g, between about 17 m²/g and about 45 m²/g, between about 18 m²/g and about 45 m²/g, between about 19 m²/g and about 45 m²/g, between about 20 m²/g and about 45 m²/g, between about 21 m²/g and about 45 m²/g, between about 22 m²/g and about 45 m²/g, between about 23 m²/g and about 45 m²/g, between about 24 m²/g and about 45 m²/g, between 3.5 m²/g and about 40 m²/g, about 4 m²/g and about 40 m²/g, about 5 m²/g and about 40 m²/g, between about 6 m²/g and about 40 m²/g, between about 7 m²/g and about 40 m²/g, between about 8 m²/g and about 40 m²/g, between about 7 m²/g and about 40 m²/g, between about 9 m²/g and about 40 m²/g, between about 10 m²/g and about 40 m²/g, between about 11 m²/g and about 40 m²/g, between about 12 m²/g and about 40 m²/g, between about 13 m²/g and about 40 m²/g, between about 14 m²/g and about 40 m²/g, between about 15 m²/g and about 40 m²/g, between about 16 m²/g and about 40 m²/g, between about 17 m²/g and about 40 m²/g, between about 18 m²/g and about 40 m²/g, between about 19 m²/g and about 40 m²/g, between about 20 m²/g and about 40 m²/g, between about 21 m²/g and about 40 m²/g, between about 22 m²/g and about 40 m²/g, between about 23 m²/g and about 40 m²/g, between about 24 m²/g and about 40 m²/g, between 3.5 m²/g and about 35 m²/g, about 4 m²/g and about 35 m²/g, about 5 m²/g and about 35 m²/g, between about 6 m²/g and about 35 m²/g, between about 7 m²/g and about 35 m²/g, between about 8 m²/g and about 35 m²/g, between about 7 m²/g and about 35 m²/g, between about 9 m²/g and about 35 m²/g, between about 10 m²/g and about 35 m²/g, between about 11 m²/g and about 35 m²/g, between about 12 m²/g and about 35 m²/g, between about 13 m²/g and about 35 m²/g, between about 14 m²/g and about 35 m²/g, between about 15 m²/g and about 35 m²/g, between about 16 m²/g and about 35 m²/g, between about 17 m²/g and about 35 m²/g, between about 18 m²/g and about 35 m²/g, between about 19 m²/g and about 35 m²/g, between about 20 m²/g and about 35 m²/g, between about 21 m²/g and about 35 m²/g, between about 22 m²/g and about 35 m²/g, between about 23 m²/g and about 35 m²/g, between about 24 m²/g and about 35 m²/g, between 3.5 m²/g and about 30 m²/g, about 4 m²/g and about 30 m²/g, about 5 m²/g and about 30 m²/g, between about 6 m²/g and about 30 m²/g, between about 7 m²/g and about 30 m²/g, between about 8 m²/g and about 30 m²/g, between about 7 m²/g and about 30 m²/g, between about 9 m²/g and about 30 m²/g, between about 10 m²/g and about 30 m²/g, between about 11 m²/g and about 30 m²/g, between about 12 m²/g and about 30 m²/g, between about 13 m²/g and about 30 m²/g, between about 14 m²/g and about 30 m²/g, between about 15 m²/g and about 30 m²/g, between about 16 m²/g and about 30 m²/g, between about 17 m²/g and about 30 m²/g, between about 18 m²/g and about 30 m²/g, between about 19 m²/g and about 30 m²/g, between about 20 m²/g and about 30 m²/g, between about 21 m²/g and about 30 m²/g, between about 22 m²/g and about 30 m²/g, between about 23 m²/g and about 30 m²/g, or between about 24 m²/g and about 30 m²/g.

In one embodiment, the cisplatin particles have a mean particle size by volume distribution (Dv50) of from about 1.0 micron to about 12.0 microns in diameter. In some embodiments, the cisplatin particles have a mean particle size by volume distribution of from about 1 micron to about 6 microns in diameter, or about 1 microns to about 3.5 or 3.0 microns in diameter. The cisplatin particles are in a size range where they are unlikely to be carried out of the tumor by systemic circulation and yet benefit from the high specific surface area to provide enhanced solubilization and release of the drug.

In one embodiment, the cisplatin particles have a mean bulk density between about 0.020 g/cm³ and about 0.8 g/cm³.

As used herein, the bulk density of the cisplatin particles is the mass of the totality of particles in the composition divided by the total volume they occupy when poured into a graduated cylinder and not tapped. The total volume includes particle volume, inter-particle void volume, and internal pore volume.

The increased specific surface area and decreased bulk density of the cisplatin particles result in significant increases in dissolution rate compared to, for example, raw or milled cisplatin products. Dissolution takes place only at a solid/liquid interface. Therefore, increased specific surface area will increase the dissolution rate due to a larger number of molecules on the surface of the particle having contact with the dissolution media. The bulk density takes into account the macrostructure and inter-particle space of a powder.

Parameters that contribute to the bulk density include particle size distribution, particle shape, and the affinity of the particles for each other (i.e., agglomeration). Lower powder bulk densities yield faster dissolution rates. This is due to the ability of the dissolution media to more readily penetrate the interstitial or inter-particle spaces and have greater contact with the surface of the particles. This provides a significant improvement for use of the cisplatin particles disclosed herein in, for example, tumor treatment.

In any of these various embodiments, the cisplatin particles may include, for example, at least 5×10⁻¹⁵ gram cisplatin per cisplatin particle, or between about 1×10⁻⁸ and about 5×10⁻¹⁵ gram cisplatin per cisplatin particle.

In one embodiment, the particles are uncoated and exclude polymer, protein, polyethoxylated castor oil and polyethylene glycol glycerides composed of mono-, di- and triglycerides and mono- and diesters of polyethylene glycol.

In a further embodiment, the composition comprises a liquid suspension further comprising a pharmaceutically acceptable liquid carrier. The suspension of the disclosure comprises cisplatin particles and a liquid carrier. The liquid carrier can be aqueous or can be non-aqueous. Even though the cisplatin particles do not include an added excipient, the liquid carrier of the suspension can comprise water or a non-aqueous liquid and optionally one or more excipients selected from the group consisting of buffer, tonicity adjusting agent, preservative, demulcent, viscosity modifier, osmotic agent, surfactant, antioxidant, alkalinizing agent, acidifying agent, antifoaming agent, and colorant. For example, the suspension can comprise cisplatin particles, water, buffer and salt. It optionally further comprises a surfactant. In some embodiments, the suspension consists essentially of or consists of water, cisplatin particles suspended in the water and buffer. The suspension can further contain an osmotic salt. In another example, the suspension can comprise cisplatin particles and a non-aqueous liquid such as a liquefied gas propellent. Examples of a liquefied gas propellent include but are not limited to hydrofluoroalkanes (HFAs). Examples of other non-aqueous liquids include but are not limited to mineral oils, vegetable oils, glycerin, polyethylene glycol, poloxamers that are liquid at room temperature (e.g., poloxamer 124), and polyethylene glycols that are liquid at room temperature, (e.g., PEG 400 and PEG 600).

In one embodiment, the suspension further comprises one or more components selected from the group consisting of polysorbate, methylcellulose, polyvinylpyrrolidone, mannitol, and hydroxypropyl methylcellulose.

The suspension can comprise one or more surfactants. Suitable surfactants include by way of example and without limitation polysorbates, lauryl sulfates, acetylated monoglycerides, diacetylated monoglycerides, and poloxamers.

The suspension can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof.

In one embodiment especially suitable for intraperitoneal (IP) administration, the suspension may be formulated to be hyperosmolar (hypertonic), hyposmolar (hypotonic) or isosmolar (isotonic) with respect to the fluid(s) of the IP cavity. In some embodiments, the suspension may be isotonic with respect to fluid in the IP cavity. In such an embodiment, the osmolality of the suspension can range from about 200 to about 380, about 240 to about 340, about 280 to about 300 or about 290 mOsm/kg.

The suspension can comprise one or more buffering agents. Suitable buffering agents include by way of example and without limitation, dibasic sodium phosphate, monobasic sodium phosphate, citric acid, sodium citrate hydrochloric acid, sodium hydroxide, tris(hydroxymethyl)aminomethane, bis(2-hydroxyethyl)iminotris-(hydroxymethyl)methane, and sodium hydrogen carbonate and others known to those of ordinary skill in the art. Buffers are commonly used to adjust the pH to a desirable range for intraperitoneal use. Usually a pH of around 5 to 9, 5 to 8, 6 to 7.4, 6.5 to 7.5, or 6.9 to 7.4 is desired.

The suspension can comprise one or more demulcents. A demulcent is an agent that forms a soothing film over a mucous membrane, such as the membranes lining the peritoneum and organs therein. A demulcent may relieve minor pain and inflammation and is sometimes referred to as a mucoprotective agent. Suitable demulcents include cellulose derivatives ranging from about 0.2 to about 2.5% such as carboxymethylcellulose sodium, hydroxyethyl cellulose, hydroxypropyl methylcellulose, and methylcellulose; gelatin at about 0.01%; polyols in about 0.05 to about 1%, also including about 0.05 to about 1%, such as glycerin, polyethylene glycol 300, polyethylene glycol 400, polysorbate 80, and propylene glycol; polyvinyl alcohol from about 0.1 to about 4%; povidone from about 0.1 to about 2%; and dextran 70 from about 0.1% when used with another polymeric demulcent described herein.

The suspension can comprise one or more alkalinizing agents to adjust the pH. As used herein, the term “alkalizing agent” is intended to mean a compound used to provide an alkaline medium. Such compounds include, by way of example and without limitation, ammonia solution, ammonium carbonate, potassium hydroxide, sodium carbonate, sodium bicarbonate, and sodium hydroxide and others known to those of ordinary skill in the art

The suspension can comprise one or more acidifying agents to adjust the pH. As used herein, the term “acidifying agent” is intended to mean a compound used to provide an acidic medium. Such compounds include, by way of example and without limitation, acetic acid, amino acid, citric acid, nitric acid, fumaric acid and other alpha hydroxy acids, hydrochloric acid, ascorbic acid, and nitric acid and others known to those of ordinary skill in the art.

The suspension can comprise one or more antifoaming agents. As used herein, the term “antifoaming agent” is intended to mean a compound or compounds that prevents or reduces the amount of foaming that forms on the surface of the fill composition. Suitable antifoaming agents include by way of example and without limitation, dimethicone, SIMETHICONE®, octoxynol and others known to those of ordinary skill in the art.

The suspension can comprise one or more viscosity modifiers that increase or decrease the viscosity of the suspension. Suitable viscosity modifiers include methylcellulose, hydroxypropyl methycellulose, mannitol and polyvinylpyrrolidone.

The suspension can comprise one or more osmotic agents such as those used for peritoneal dialysis. Suitable osmotic agents include icodextrin (a glucose polymer), sodium chloride, potassium chloride, and salts that are also used as buffering agents.

In one embodiment, a liquid suspension of the cisplatin particles may be aerosolized for pulmonary administration by inhalation, and the mass median aerodynamic diameter (MMAD) of the aerosol droplets of the liquid suspension may be any suitable diameter for use. In one embodiment, the aerosol droplets have a MMAD of between about 0.5 μm to about 6 μm diameter. In various further embodiments, the aerosol droplets have a MMAD of between about 0.5 μm to about 5.5 μm diameter, about 0.5 μm to about 5 μm diameter, about 0.5 μm to about 4.5 μm diameter, about 0.5 μm to about 4 μm diameter, about 0.5 μm to about 3.5 μm diameter, about 0.5 μm to about 3 μm diameter, about 0.5 μm to about 2.5 μm diameter, about 0.5 μm to about 2 μm diameter, about 1 μm to about 5.5 μm diameter, about 1 μm to about 5 μm diameter, about 1 μm to about 4.5 μm diameter, about 1 μm to about 4 μm diameter, about 1 μm to about 3.5 μm diameter, about 1 μm to about 3 μm diameter, about 1 μm to about 2.5 μm diameter, about 1 μm to about 2 μm diameter, about 1.5 μm to about 5.5 μm diameter, about 1.5 μm to about 5 μm diameter, about 1.5 μm to about 4.5 μm diameter, about 1.5 μm to about 4 μm diameter, about 1.5 μm to about 3.5 μm diameter, about 1.5 μm to about 3 μm diameter, about 1.5 μm to about 2.5 μm diameter, about 1.5 μm to about 2 μm diameter, about 2 μm to about 5.5 μm diameter, about 2 μm to about 5 μm diameter, about 2 μm to about 4.5 μm diameter, about 2 μm to about 4 μm diameter, about 2 μm to about 3.5 μm diameter, about 2 μm to about 3 μm diameter, and about 2 μm to about 2.5 μm diameter. A suitable instrument for measuring the mass median acrodynamic diameter (MMAD) and geometric standard deviation (GSD) of the aerosol droplets is a seven-stage aerosol sampler such as the Mercer-Style Cascade Impactor. Liquid suspensions of cisplatin particles delivered by aerosol may be deposited in the airways by gravitational sedimentation, inertial impaction, and/or diffusion. Any suitable device for generating the aerosol may be used, including but not limited to metered dose inhalers (MDI), pressured metered dose inhalers (pMDI), nebulizers, and soft-mist inhalers.

In one embodiment, a dry powder composition of cisplatin particles may be aerosolized for pulmonary administration by inhalation, and the mass median aerodynamic diameter (MMAD) of the aerosolized dry powder composition may be any suitable diameter for use. The dry powder composition is formulated as a dry powder. The dry powder composition can contain cisplatin particles alone without a carrier or can comprise cisplatin particles and a pharmaceutically acceptable dry powder carrier comprising one or more dry powder excipients. In one embodiment, the aerosolized dry powder composition has a MMAD of between about 0.5 μm to about 6 μm diameter. In various further embodiments, the aerosolized dry powder composition has a MMAD of between about 0.5 μm to about 5.5 μm diameter, about 0.5 μm to about 5 μm diameter, about 0.5 μm to about 4.5 μm diameter, about 0.5 μm to about 4 μm diameter, about 0.5 μm to about 3.5 μm diameter, about 0.5 μm to about 3 μm diameter, about 0.5 μm to about 2.5 μm diameter, about 0.5 μm to about 2 μm diameter, about 1 μm to about 5.5 μm diameter, about 1 μm to about 5 μm diameter, about 1 μm to about 4.5 μm diameter, about 1 μm to about 4 μm diameter, about 1 μm to about 3.5 μm diameter, about 1 μm to about 3 μm diameter, about 1 μm to about 2.5 μm diameter, about 1 μm to about 2 μm diameter, about 1.5 μm to about 5.5 μm diameter, about 1.5 μm to about 5 μm diameter, about 1.5 μm to about 4.5 μm diameter, about 1.5 μm to about 4 μm diameter, about 1.5 μm to about 3.5 μm diameter, about 1.5 μm to about 3 μm diameter, about 1.5 μm to about 2.5 μm diameter, about 1.5 μm to about 2 μm diameter, about 2 μm to about 5.5 μm diameter, about 2 μm to about 5 μm diameter, about 2 μm to about 4.5 μm diameter, about 2 μm to about 4 μm diameter, about 2 μm to about 3.5 μm diameter, about 2 μm to about 3 μm diameter, and about 2 μm to about 2.5 μm diameter. A suitable instrument for measuring the mass median aerodynamic diameter (MMAD) and geometric standard deviation (GSD) of the dry powder composition is a seven-stage aerosol sampler such as the Mercer-Style Cascade Impactor or an acrodynamic particle sizer spectrometer such as the APS™ Model 3321 spectrometer available from TSI Incorporated. The dry powder composition delivered by aerosol may be deposited in the airways by gravitational sedimentation, inertial impaction, and/or diffusion. Any suitable device for generating the aerosol of the dry powder composition may be used, including but not limited to dry powder inhalers (DPI). An example of an excipient suitable for a dry powder inhalable composition includes but is not limited to lactose in grades suitable for inhalation. In one embodiment, the composition is a dry powder composition suitable for pulmonary delivery by inhalation via aerosolization.

In one embodiment, the composition comprises a dosage form of cisplatin in suspension (i.e.: with a pharmaceutically acceptable carrier and any other components), in a dosage deemed suitable by an attending physician for an intended use. Any suitable dosage form may be used; in various non-limiting embodiments, the dosage form is adequate to provide about 0.01 mg/kg to about 50 mg/kg of body weight per day. In various further embodiments, the dosage form is adequate to provide about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg of body weight per day. The suspension can be administered as is or can be diluted with a diluent.

In another aspect, the disclosure provides methods for treating a tumor, comprising administering to a subject with a tumor an amount effective to treat the tumor of the composition or suspension of any embodiment or combination of embodiments of the disclosure. The increased specific surface area of the cisplatin particles of the disclosure results in the significant increase in dissolution rate for the particles compared to currently available cisplatin. This provides a significant improvement for use of the particles of the disclosure in, for example, tumor treatment. Furthermore, in some embodiments the methods of the present disclosure can reduce the dosing frequency and side effects of cisplatin. By way of non-limiting example, a cisplatin dose administered by direct tumoral injection would provide significant benefits, with reduced side effects, since the systemic concentrations would be greatly reduced. Cisplatin particles of the disclosure dissolving inside of a tumor create higher concentrations of dissolved cisplatin compared to the concentrations of cisplatin in the surrounding fluid. The localized depot of higher cisplatin concentrations interacts with the rapidly dividing tumor cells to a greater extent compared to cisplatin delivered to the tumor systemically. This reduces the cellular interactions of cisplatin outside the tumor. The higher surface area of the particles decreases the time needed to achieve the higher localized concentration of cisplatin inside the tumor.

As used herein, a “tumor” includes benign tumors, pre-malignant tumors, malignant tumors that have not metastasized, and malignant tumors that have metastasized. The methods of the disclosure can be used to treat tumor that is susceptible to cisplatin treatment, including but not limited to carcinomas, breast tumors, pancreatic tumors, prostate tumors, bladder tumors, lung tumors, ovarian tumors, gastrointestinal tumors, testicular tumors, cervical tumors, head and neck tumors, esophageal tumors, mesothelioma, brain tumors, neuroblastomas, or renal cell tumors. In specific embodiments, the tumor is a metastatic testicular tumor, metastatic ovarian tumor, or advanced bladder cancer.

In another embodiment, the method further comprises administering an additional therapeutic to the subject, including but not limited to anthracyclines, antimetabolites, alkylating agents, alkaloids, taxanes (including but not limited to paclitaxel, docetaxel, cabazitaxel, and combinations thereof), and/or topoisomerase inhibitors.

In specific embodiments, the one or more additional therapeutics may comprise one or more of durvalumab, tremelimumab, and/or etoposide.

The subject may be any suitable subject with a tumor, including but not limited to humans, primates, dogs, cats, horses, cattle, etc. In one embodiment, the subject is a human subject.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (c) limiting or preventing recurrence of symptoms in patients that were previously symptomatic for the disorder(s).

Amounts effective for these uses depend on factors including, but not limited to, the nature of the cisplatin (specific activity, etc.), the route of administration, the stage and severity of the disorder, the weight and general state of health of the subject, and the judgment of the prescribing physician. It will be understood that the amount of the composition of suspension of the disclosure actually administered will be determined by a physician, in the light of the above relevant circumstances. In one non-limiting embodiment, an amount effective is an amount that provides between 0.01 mg/kg to about 50 mg/kg of body weight per day.

The compositions may be administered via any suitable route, including but not limited to orally, pulmonary, intraperitoneally, intra-tumorally, peri-tumorally, subcutaneous injection, intramuscular injection, administered into a mammary fat pad, or any other form of injection, as deemed most appropriate by attending medical personnel in light of all factors for a given subject.

In one embodiment, pulmonary administration comprises inhalation of a single dose of the cisplatin particles, such as by nasal, oral inhalation, or both. The cisplatin particles can be administered in two or more separate administrations (doses). In this embodiment, the particles may be formulated as an aerosol (i.e.: liquid droplets of a stable dispersion or suspension of the particles in a gaseous medium). Cisplatin particles delivered by aerosol may be deposited in the airways by gravitational sedimentation, inertial impaction, and/or diffusion. Any suitable device for generating the aerosol may be used, including but not limited to metered dose inhalers (MDI), pressured metered dose inhalers (pMDI), nebulizers, and soft-mist inhalers.

In one specific embodiment, the methods comprise inhalation of cisplatin particles aerosolized via nebulization. Nebulizers generally use compressed air or ultrasonic power to create inhalable aerosol droplets of the particles or suspensions thereof. In this embodiment, the nebulizing results in pulmonary delivery to the subject of aerosol droplets of the cisplatin particles or suspension thereof.

In another embodiment, the methods comprise inhalation of cisplatin particles aerosolized via a pMDI, wherein the particles or suspensions thereof are suspended in a suitable propellant system (including but not limited to hydrofluoroalkanes (HFAs) containing at least one liquefied gas in a pressurized container sealed with a metering valve. Actuation of the valve results in delivery of a metered dose of an aerosol spray of the cisplatin particles or suspensions thereof.

In another embodiment, the methods comprise inhalation of a dry powder composition of cisplatin via a DPI, wherein the dry powder composition contains cisplatin particles alone without a carrier. In still another embodiment, the methods comprise inhalation of a dry powder composition of cisplatin via a DPI, wherein the dry powder composition comprises cisplatin particles and may include a pharmaceutically acceptable dry powder carrier comprising one or more dry powder excipients. An example of a dry powder excipient suitable for a dry powder inhalable composition includes but is not limited to lactose in grades suitable for inhalation.

A dosing period is that period of time during which a dose of cisplatin particles in the composition or suspension is administered. The dosing period can be a single period of time during which the entire dose is administered, or it can be divided into two or more periods of time during each of which a portion of the dose is administered.

A post-dosing period is that period of time beginning after completion of a prior dosing period and ending after initiating a subsequent dosing period. The duration of the post-dosing period may vary according to a subject's clinical response to the cisplatin. The suspension is not administered during the post-dosing period. A post-dosing period can last at least 7 days, at least 14 days, at least 21 days, at least 28 days, at least 35 days, at least 60 days or at least 90 days or longer. The post-dosing period can be kept constant for a subject or two or more different post-dosing periods can be used for a subject.

A dosing cycle includes a dosing period and a post-dosing period. Accordingly, the duration of a dosing cycle will be the sum of the dosing period and the post-dosing period. The dosing cycle can be kept constant for a subject or two or more different dosing cycles can be used for a subject.

In one embodiment, the administering is carried out more than once, and wherein each administration is separated in time by at least 21 days.

In another aspect, the disclosure provides methods for making cisplatin particles, comprising:
(a) introducing (i) a solution comprising at least one solvent including but not limited to DMF (dimethylformamide), DMSO (dimethyl sulfoxide), acetone, or combinations thereof, or combinations thereof, and at least one solute comprising cisplatin into a nozzle inlet, and (ii) a compressed fluid into an inlet of a vessel defining a pressurizable chamber;
(b) passing the solution out of a nozzle orifice and into the pressurizable chamber to produce an output stream of atomized droplets, wherein the nozzle orifice is located between 2 mm and 20 mm from a sonic energy source located within the output stream, wherein the sonic energy source produces sonic energy with an amplitude between 10% and 100% during the passing, and wherein the nozzle orifice has a diameter of between 20 μm and 125 μm;
(c) contacting the atomized droplets with the compressed fluid, to cause depletion of the solvent from the atomized droplets, to produce cisplatin particles comprising at least 95% cisplatin, wherein the cisplatin particles have a specific surface area (SSA) of at least 3.5 m²/g and have a mean particle size of between about 0.7 μm and about 8 μm,
wherein steps (a), (b), and (c) are carried out under supercritical temperature and pressure for the compressed fluid.

The methods utilize a sonic energy source located directly in the output stream of the solute dissolved in the solvent. Any suitable source of sonic energy may be used that is compatible with the methods of the disclosure, including but not limited to sonic horn, a sonic probe, or a sonic plate. In various embodiments, the nozzle orifice is located between about 2 mm and about 20 mm, about 2 mm and about 18 mm, about 2 mm and about 16 mm, about 2 mm and about 14 mm, about 2 mm and about 12 mm, about 2 mm and about 10 mm, about 2 mm and about 8 mm, about 2 mm and about 6 mm, about 2 mm and about 4 mm, about 4 mm and about 20 mm, about 4 mm and about 18 mm, about 4 mm and about 16 mm, about 4 mm and about 14 mm, about 4 mm and about 12 mm, about 4 mm and about 10 mm, about 4 mm and about 8 mm, about 4 mm and about 6 mm, about 6 mm and about 20 mm, about 6 mm and about 18 mm, about 6 mm and about 16 mm, about 6 mm and about 14 mm, about 6 mm and about 12 mm, about 6 mm and about 10 mm, about 6 mm and about 8 mm, about 8 mm and about 20 mm, about 8 mm and about 18 mm, about 8 mm and about 16 mm, about 8 mm and about 14 mm, about 8 mm and about 12 mm, about 8 mm and about 10 mm, about 10 mm and about 20 mm, about 10 mm and about 18 mm, about 10 mm and about 16 mm, about 10 mm and about 14 mm, about 10 mm and about 12 mm, about 12 mm and about 20 mm, about 12 mm and about 18 mm, about 12 mm and about 16 mm, about 12 mm and about 14 mm, about 14 mm and about 20 mm, about 14 mm and about 18 mm, about 14 mm and about 16 mm, about 16 mm and about 20 mm, about 16 mm and about 18 mm, and about 18 mm and about 20 mm, from the sonic energy source. In further embodiments, the nozzle assembly of any embodiment of WO2016/197091 may be used.

Any suitable source of sonic energy may be used that is compatible with the methods of the disclosure, including but not limited to sonic horn, a sonic probe, or a sonic plate. In various further embodiments, the sonic energy source produces sonic energy with an amplitude between about 10% and about 100% of the total power that can be generated using the sonic energy source. In light of the teachings herein, one of skill in the art can determine an appropriate sonic energy source having a specific total power output to be used. In one embodiment, the sonic energy source has a total power output of between about 500 and about 900 watts; in various further embodiments, between about 600 and about 800 watts, about 650-750 watts, or about 700 watts.

In various further embodiments, the sonic energy source produces sonic energy with a power output between about 20% and about 100%, about 30% and about 100%, about 40% and about 100%, about 50% and about 100%, about 60% and about 100%, about 70% and about 100%, about 80% and about 100%, about 90% and about 100%, about 10% and about 90%, about 20% and about 90%, about 30% and about 90%, about 40% and about 90%, about 50% and about 90%, about 60% and about 90%, about 70% and about 90%, about 80% and about 90%, about 10% and about 80%, about 20% and about 80%, about 30% and about 80%, about 40% and about 80%, about 50% and about 80%, about 60% and about 80%, about 70% and about 80%, about 10% and about 70%, about 20% and about 70%, about 30% and about 70%, about 40% and about 70%, about 50% and about 70%, about 60% and about 70%, about 10% and about 60%, about 20% and about 60%, about 30% and about 60%, about 40% and about 60%, about 50% and about 60%, about 10% and about 50%, about 20% and about 50%, about 30% and about 50%, about 40% and about 50%, about 10% and about 40%, about 20% and about 40%, about 30% and about 40%, about 10% and about 30%, about 20% and about 30%, about 10% and about 20%, or about 10%, 20%, 30%, 40%, 50%, 60%, 70%, 80%, 90%, or about 100% of the total power that can be generated using the sonic energy source. In light of the teachings herein, one of skill in the art can determine an appropriate frequency to be utilized on the sonic energy source. In one embodiment, a frequency of between about 18 and about 22 kHz on the sonic energy source is utilized. In various other embodiments, a frequency of between about 19 and about 21 kHz, about 19.5 and about 20.5, or a frequency of about 20 kHz on the sonic energy source is utilized.

In various further embodiments, the nozzle orifice has a diameter of between about 20μm and about 125 μm, about 20 μm and about 115 μm, about 20 μm and about 100 μm, about 20 μm and about 90 μm, about 20 μm and about 80 μm, about 20 μm and about 70 μm, about 20 μm and about 60 μm, about 20 μm and about 50 μm, about 20 μm and about 40 μm, about 20 μm and about 30 μm, between about 30 μm and about 125 μm, about 30 μm and about 115 μm, about 30 μm and about 100 μm, about 30 μm and about 90 μm, about 30 μm and about 80 μm, about 30 μm and about 70 μm, about 30 μm and about 60 μm, about 30 μm and about 50 μm, about 30 μm and about 40 μm, between about 40 μm and about 125 μm, about 40 μm and about 115 μm, about 40 μm and about 100 μm, about 40 μm and about 90 μm, about 40 μm and about 80 μm, about 40 μm and about 70 μm, about 40 μm and about 60 μm, about 40 μm and about 50 μm, between about 50 μm and about 125 μm, about 50 μm and about 115 μm, about 50 μm and about 100 μm, about 50 μm and about 90 μm, about 50 μm and about 80 μm, about 50 μm and about 70 μm, about 50 μm and about 60 μm, between about 60 μm and about 125 μm, about 60 μm and about 115 μm, about 60 μm and about 100 μm, about 60 μm and about 90 μm, about 60 μm and about 80 μm, about 60 μm and about 70 μm, between about 70 μm and about 125 μm, about 70 μm and about 115 μm, about 70 μm and about 100 μm, about 70 μm and about 90 μm, about 70 μm and about 80 μm, between about 80 μm and about 125 μm, about 80 μm and about 115 μm, about 80 μm and about 100 μm, about 80 μm and about 90 μm, between about 90 μm and about 125 μm, about 90 μm and about 115 μm, about 90 μm and about 100 μm, between about 100 μm and about 125 μm, about 100 μm and about 115 μm, between about 115 μm and about 125 μm, about 20 μm, 30 μm, 40 μm, 50 μm, 60 μm, 70 μm, 80 μm, 90 μm, 100 μm, 115 μm, or about 120 μm. The nozzle is inert to both the solvent and the compressed fluid used in the methods.

The solvent comprises DMF (dimethylformamide), DMSO (dimethyl sulfoxide), acetone, or combinations thereof. The solvent should comprise at least about 80%, 85%, or 90% by weight of the overall solution.

The compressed fluid is capable of forming a supercritical fluid under the conditions used, and the solute that forms the particles is poorly soluble or insoluble in the compressed fluid. As is known to those of skill in the art, a supercritical fluid is any substance at a temperature and pressure above its critical point, where distinct liquid and gas phases do not exist. Steps (a), (b), and (c) of the methods of the disclosure are carried out under supercritical temperature and pressure for the compressed fluid, such that the compressed fluid is present as a supercritical fluid during these processing steps.

The compressed fluid can serve as a solvent for and can be used to remove unwanted components in the particles. Any suitable compressed fluid may be used in the methods of the disclosure; exemplary such compressed fluids are disclosed in U.S. Pat. Nos. 5,833,891 and 5,874,029. In one non-limiting embodiment, suitable supercritical fluid-forming compressed fluids and/or anti-solvents can comprise carbon dioxide, ethane, propane, butane, isobutane, nitrous oxide, xenon, sulfur hexafluoride and trifluoromethane. The anti-solvent recited in step (d) to cause further solvent depletion, is a compressed fluid as defined above, and may be the same compressed fluid used in steps (a-c), or may be different. In one embodiment, the anti-solvent used in step (d) is the same as the compressed fluid used in steps (a-c). In a preferred embodiment, the compressed fluid and the anti-solvent are both super-critical carbon dioxide.

In all cases, the compressed fluid and anti-solvent should be substantially miscible with the solvent while the cisplatin should be substantially insoluble in the compressed fluid, i.e., cisplatin, at the selected solvent/compressed fluid contacting conditions, should be no more than about 5% by weight soluble in the compressed fluid or anti-solvent, and preferably is essentially completely insoluble.

The supercritical conditions used in the methods of the disclosure are typically in the range of from 1X to about 1.4X, or 1X to about 1.2X of the critical temperature of the supercritical fluid, and from 1X to about 7X, or 1X to about 2X, of the of the supercritical pressure for the compressed fluid. It is well within the level of those of skill in the art to determine the critical temperature and pressure for a given compressed fluid or anti-solvent. In one embodiment, the compressed fluid and anti-solvent are both super critical carbon dioxide, and the critical temperature is at least 31.1° C. and up to about 60° C., and the critical pressure is at least 1071 psi and up to about 1800 psi. In another embodiment, the compressed fluid and anti-solvent are both super critical carbon dioxide, and the critical temperature is at least 35° C. and up to about 55° C., and the critical pressure is at least 1070 psi and up to about 1500 psi. It will be understood by those of skill in the art that the specific critical temperature and pressure may be different at different steps during the processing.

Any suitable pressurizable chamber may be used, including but not limited to those disclosed in WO2016/197091 or in U.S. Pat. Nos. 5,833,891 and 5,874,029. Similarly, the steps of contacting the atomized droplets with the compressed fluid to cause depletion of the solvent from the droplets; and contacting the droplets with an anti-solvent to cause further depletion of the solvent from the droplets, to produce particles of the compound can be carried out under any suitable conditions, including but not limited to those disclosed in U.S. Pat. Nos. 5,833,891 and 5,874,029.

The flow rate can be adjusted as high as possible to optimize output but below the pressure limitations for the equipment, including the nozzle orifice. In one embodiment, the flow rate of the solution through the nozzle has a range from about 0.5 mL/min to about 30 mL/min. In various further embodiments, the flow rate is between about 0.5 mL/min to about 25 ml/min, 0.5 mL/min to about 20 mL/min, 0.5 mL/min to about 15 ml/min, 0.5 mL/min to about 10 mL/min, 0.5 mL/min to about 4 mL/min, about 1 mL/min to about 30 mL/min, about 1 mL/min to about 25 mL/min, about 1 mL/min to about 20 mL/min, 1 mL/min to about 15 mL/min, about 1 mL/min to about 10 mL/min, about 2 mL/min to about 30 mL/min, about 2 mL/min to about 25 mL/min, about 2 mL/min to about 20 mL/min, about 2 mL/min to about 15 mL/min, or about 2 mL/min to about 10 mL/min. The solution of drug subject to the flow rate can be any suitable concentration, such as between about 1 mg/ml and about 80 mg/ml.

In one embodiment, the methods further comprise receiving the plurality of particles through the outlet of the pressurizable chamber; and collecting the plurality of particles in a collection device, such as disclosed in WO2016/197091.

In another aspect, the disclosure provides cisplatin particles prepared by the method of any embodiment or combination of embodiments of the disclosure.

### EXAMPLES

### Description of Substances

Compound Name: Cisplatin
Molecular Formula: Pt(NH₃)₂Cl₂
Molecular Weight: 300.05 g/mole

### Material Testing

Particle Size Distribution (PSD) by Laser Diffraction
Imaging by Scanning Electron Microscopy (SEM)
Specific Surface Area (SSA) determination by BET Sorptometry
Crystalline/amorphous phase determination by Powder X-ray Diffraction (PXRD)
Bulk density analysis by modified USP <616> Bulk Density of Powders Method I

### Study

1. Solvent solubility testing of cisplatin in various solvents.
2. Demonstrate precipitating cisplatin from three solvent systems, then analyzing the corresponding materials for/by PSD, SEM, PXRD, SSA, and bulk density.

The solubility of cisplatin was tested in the following solvent mixtures:
1:3 DMSO: Acetone,
1:1 DMSO: Acetone,
3:1 DMSO: Acetone,
DMF alone, and
DMSO alone.

Three small scale precipitation runs were conducted with cisplatin on an RC612B precipitation unit. The precipitates from the runs were analyzed by laser diffraction to determine PSD, SEM to support PSD data and determine shape/habit, BET sorptometry to determine the SSA, PXRD to determine crystalline/amorphous phase of the material, and bulk density analysis to identify additional physical characteristics of the precipitates.

### Experimental Procedures

### Material Receipt

Cisplatin was obtained from BOC Sciences and stored in a temperature and humidity monitored cabinet.

### Solvent Selection

Solubility in an organic solvent greater than ˜8 mg/mL at room temperature was deemed adequate for further studies, with greater solvent solubility resulting in a decreased production time. Solubility was determined by visual observation and tested according to standard operating procedure.

### Precipitation

Thirteen small-scale precipitates of cisplatin were produced on the RC612B SCP unit according to standard operating procedure EQP-002, Operation, Maintenance, and Calibration of RC612B.

In one particular exemplary method, a solution of 16.8 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 38° C. and a flow rate of 4 to 12 kg/hour. The sonic probe was adjusted to an amplitude of 60% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 100.4 mg/mL of cisplatin was prepared in DMSO. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 38° C. and a flow rate of 4 to 12 kg/hour. The sonic probe was adjusted to an amplitude of 60% of maximum output at a frequency of 20 kHz. The DMSO solution containing the cisplatin was pumped through the nozzle at a flow rate of 2 mL/minute for approximately 3 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 49.7 mg/mL of cisplatin was prepared in 3:2 (v/v) DMSO: Acetone. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 38° C. and a flow rate of 4 to 12 kg/hour. The sonic probe was adjusted to an amplitude of 60% of maximum output at a frequency of 20 kHz. The 3:2 DMSO: Acetone solution containing the cisplatin was pumped through the nozzle at a flow rate of 2 mL/minute for approximately 5 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.7 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1300 psi at about 39° C. and a flow rate of 4 to 12 kg/hour. The sonic probe was adjusted to an amplitude of 60% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.8 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1100 psi at about 38° C. and a flow rate of 4 to 12 kg/hour. The sonic probe was adjusted to an amplitude of 60% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.8 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 37° C. and a flow rate of 4 to 12 kg/hour. The sonic probe was adjusted to an amplitude of 60% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.8 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 42° C. and a flow rate of 4 to 12 kg/hour. The sonic probe was adjusted to an amplitude of 60% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.7 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 39° C. and a flow rate of 4 to 12 kg/hour. The sonic probe was adjusted to an amplitude of 20% of maximum output at a frequency of 20 KHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.8 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 38° C. and a flow rate of 4 to 12 kg/hour. The sonic probe was adjusted to an amplitude of 80% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.7 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 37° C. and a flow rate of 4 to 12 kg/hour. The sonic probe was adjusted to an amplitude of 0% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

In another exemplary method, a solution of 16.7 mg/mL of cisplatin was prepared in DMF. The nozzle and a sonic probe were positioned in the pressurizable chamber approximately 9 mm apart. A stainless steel membrane filter with approximately 20 nm nominal rating was attached to the pressurizable chamber to collect the precipitated cisplatin particles. The supercritical carbon dioxide was placed in the pressurizable chamber of the manufacturing equipment and brought to approximately 1200 psi at about 36° C. and a flow rate of 4 to 12 kg/hour. The sonic probe was adjusted to an amplitude of 20% of maximum output at a frequency of 20 kHz. The DMF solution containing the cisplatin was pumped through the nozzle at a flow rate of 2 mL/minute for approximately 15 minutes. The precipitated cisplatin particles were then collected from the supercritical carbon dioxide as the mixture was pumped through the stainless steel mesh filter. The filter containing the particles of cisplatin was opened and the resulting product was collected from the filter.

### Analytical Testing

Following the three precipitation runs of cisplatin, the materials were analyzed for/by PSD, SEM, PXRD, SSA, and bulk density where applicable.

### Results and Discussion

### Precipitation

The first and second precipitation runs were conducted with DMF (SC1) and DMSO (SC2), respectively. The final run (SC3) was conducted from a 3:2 DMSO: Acetone mixture to achieve a 50 mg/mL concentration. SC1 resulted in yield of 85.6%, which is a good yield at small scale. SC2 resulted in a yield of 54.1% which is clearly lower than SC1, but an acceptable yield at small scale. SC3 resulted in an 18.6% yield.

### Particle Size Distribution

Particle size analyses were conducted on a Malvern Mastersizer™ 3000 using the Hydro MV dispersion unit. A non-validated general PSD method/dispersant method was used to analyze the cisplatin samples. The sample preparation procedure performed was as follows: Weigh 10 to 20 mg of cisplatin into a 30-mL vial, add 20 mL of ethyl acetate. Disperse the sample by vortexing then sonicating the suspension in an ultrasonic bath for 1 minute. Then transfer the sample suspension to the Malvern Hydro MV dispersion unit to obtain obscuration between 5 and 15%.

PSD results from SC1 and SC3 were relatively similar, with the only difference between SC1 and SC3 being the Dv90. SC1 and SC3 showed significant decreases in PSD when compared to the raw material.

### Scanning Electron Microscopy

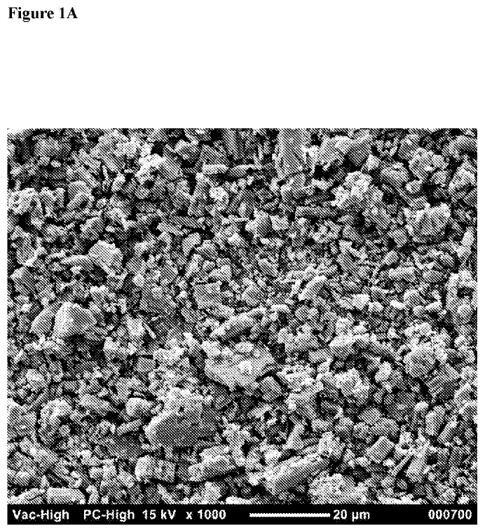

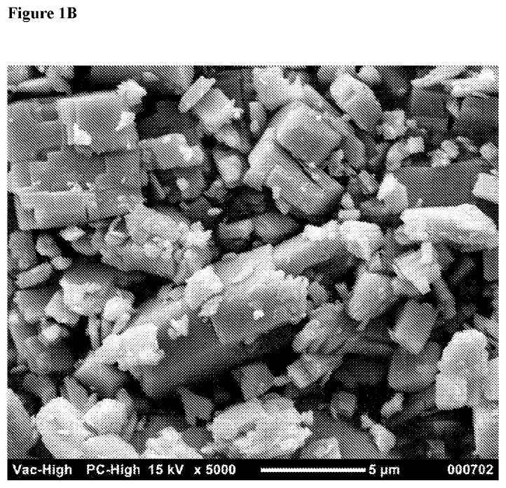

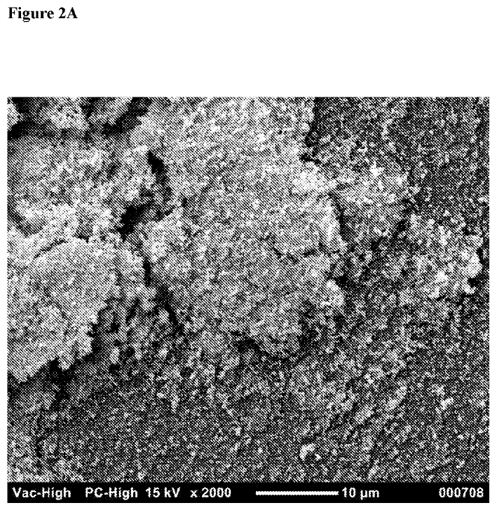

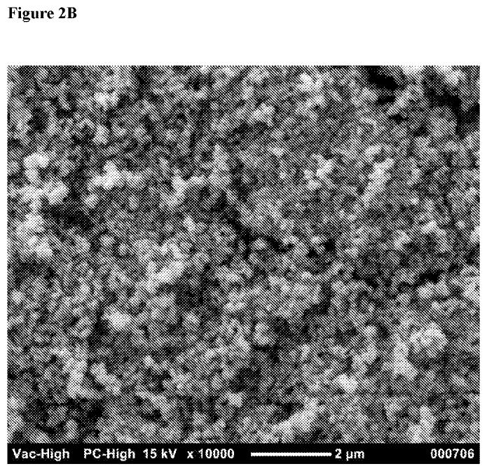

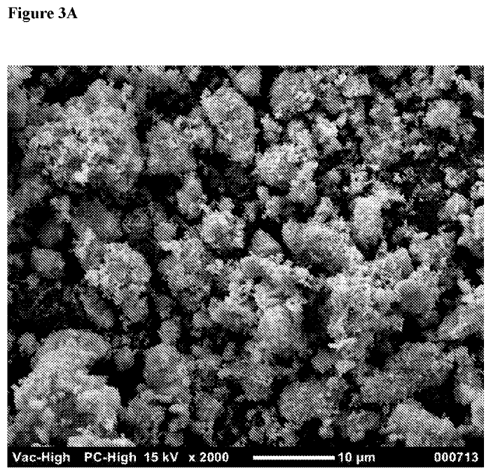

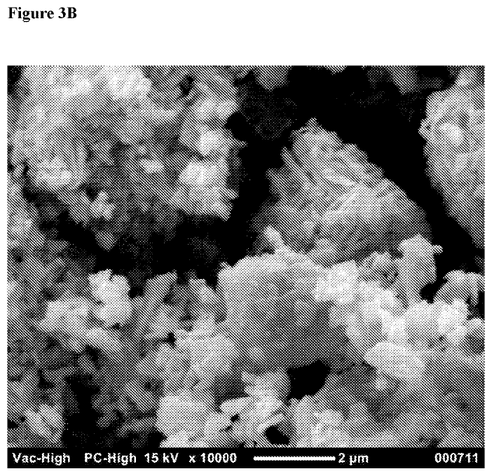

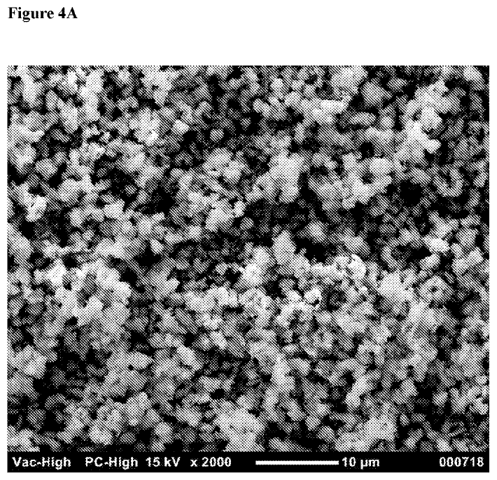

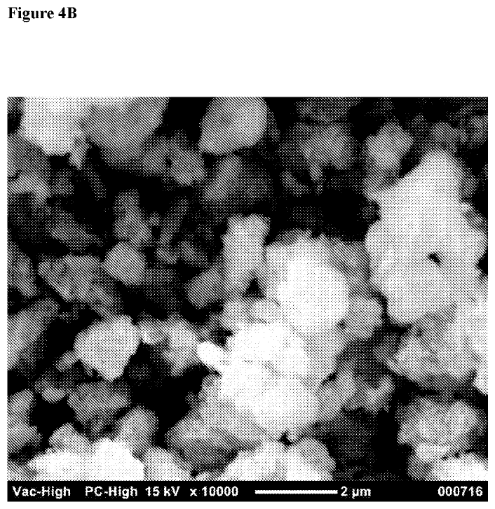

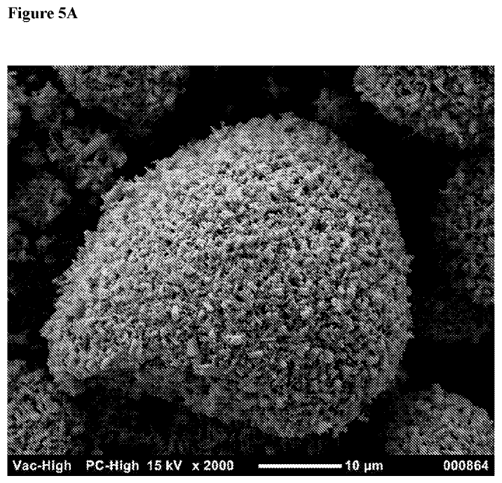

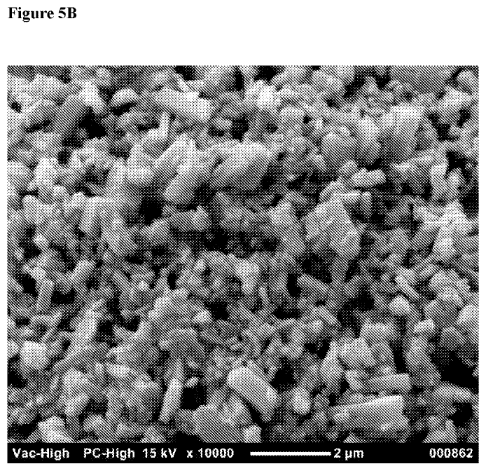

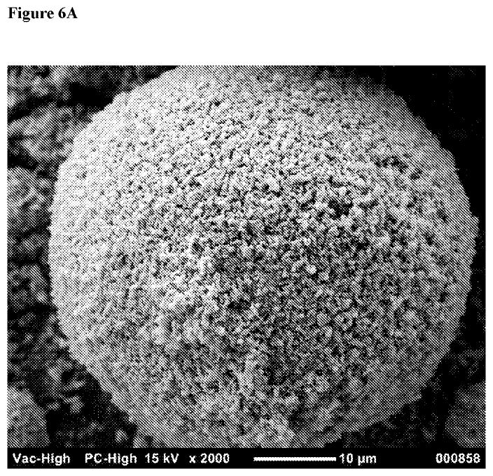

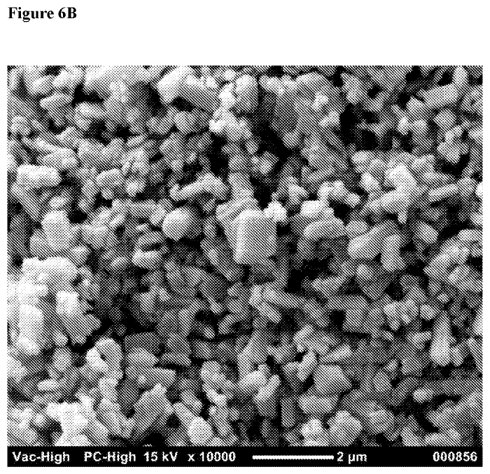

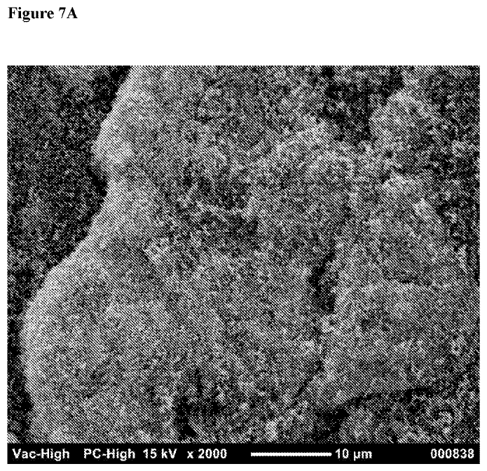

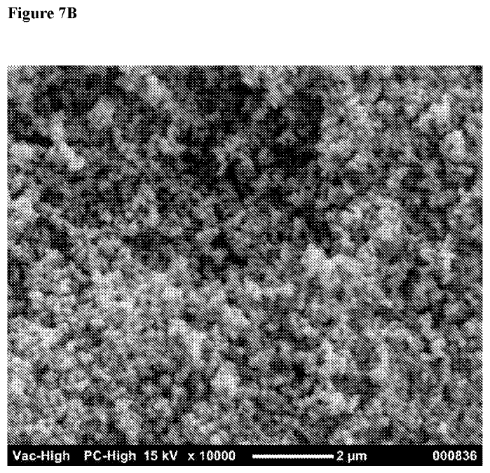

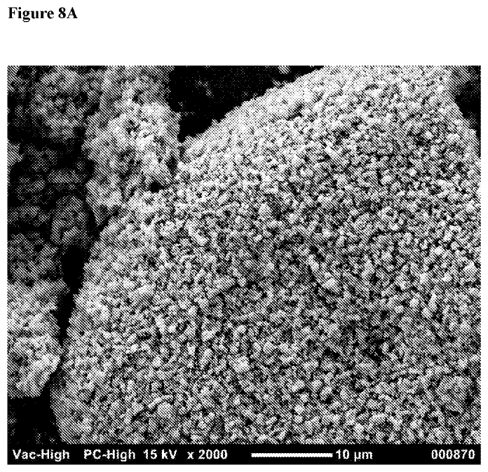

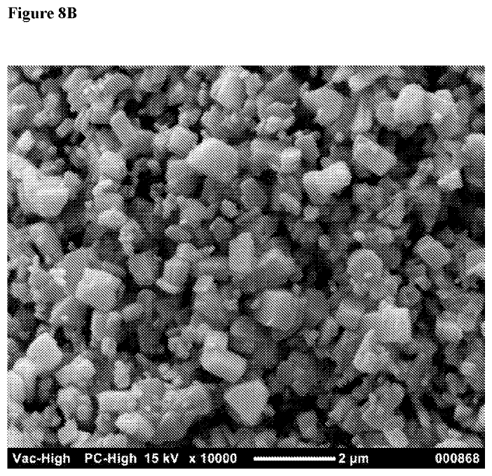

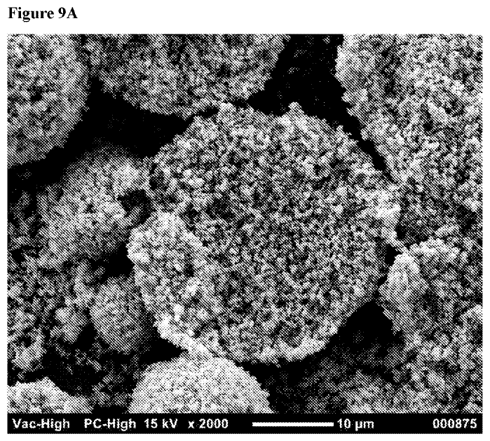

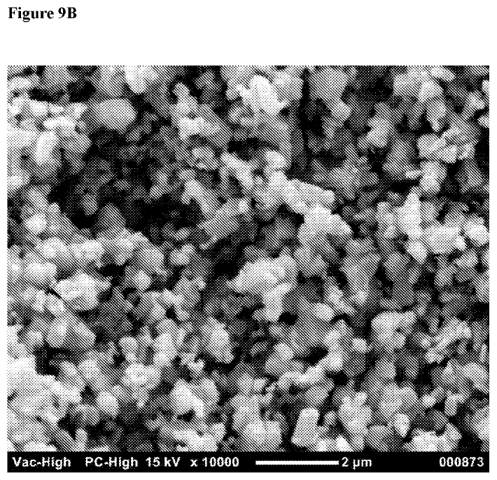

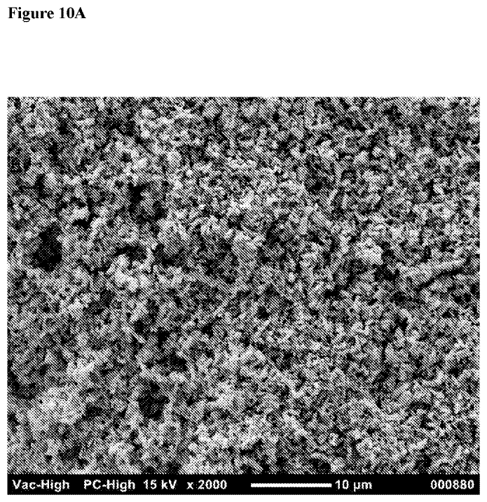

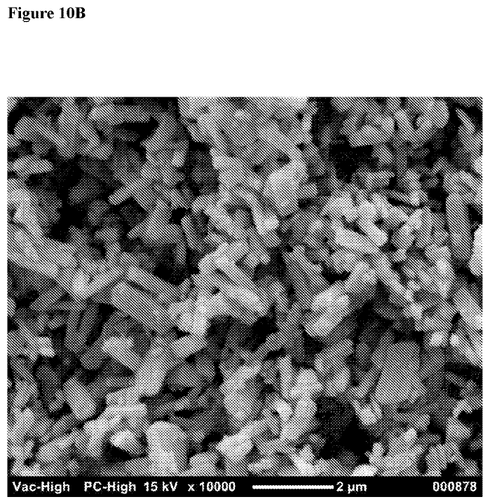

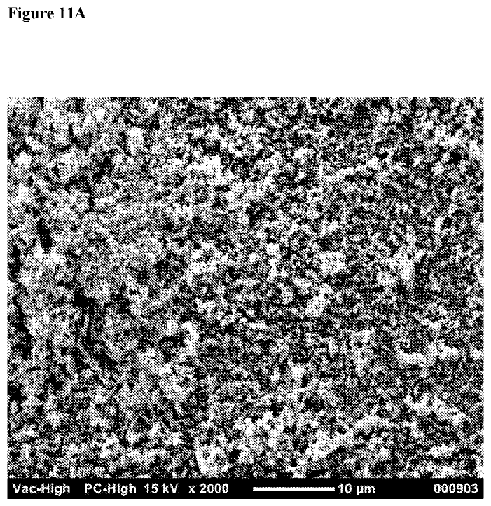

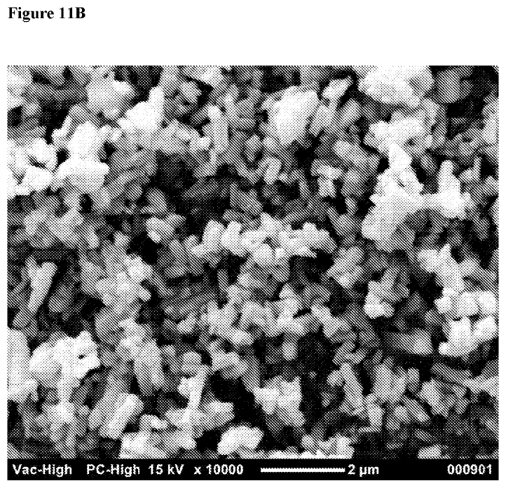

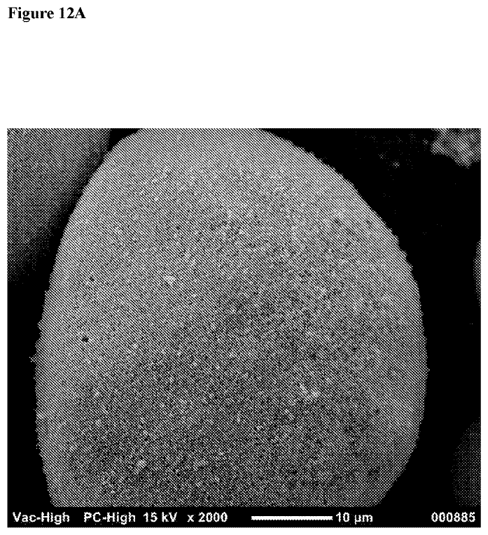

Scanning electron microscopy was conducted on a Joel NeoScope™ SEM. Overall, imaging supports the particle size distribution results with varying distributions, as well as different particle shapes/habits. SEM micrographs of SC1 and SC2 exhibited particles in the <1 μm range, which were observed in the PSD results, but not at the level anticipated based on SEM. PSD method development would help clarify whether the method used lacked sufficient dispersion energy necessary to break up agglomerates or if the Dv90 of 9.18 and 10.83 μm are true and the material has aggregated into fused larger particles. SEM micrographs are shown in FIGS. 1-14; the solvents used and magnifications are shown in the corresponding figure legends.

### Powder X-ray Diffraction

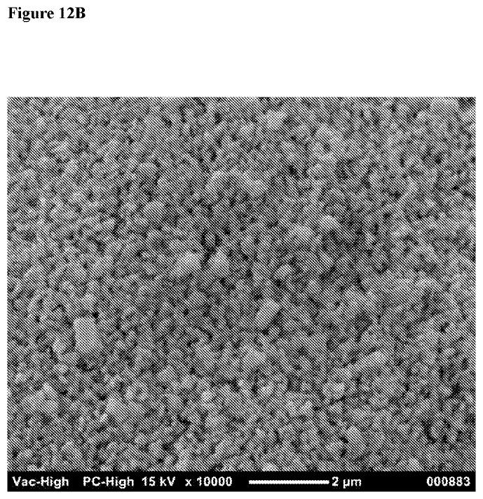

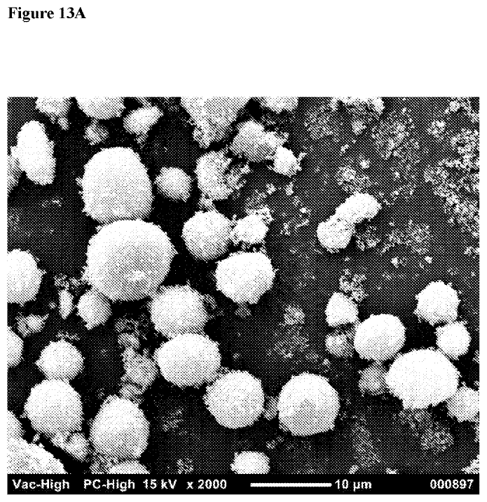

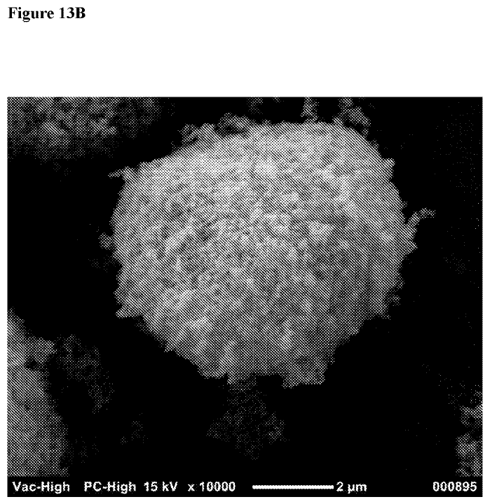

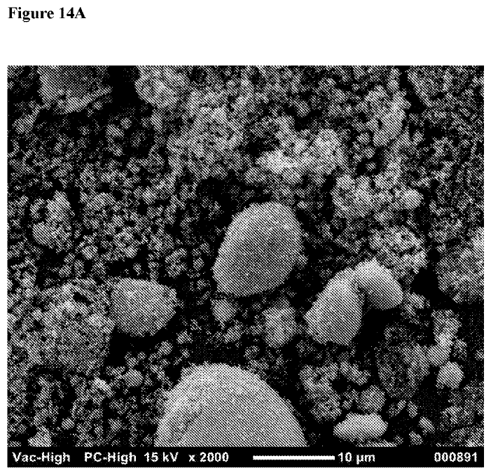

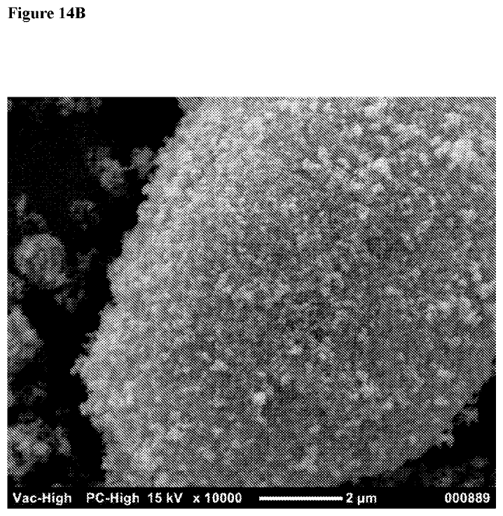

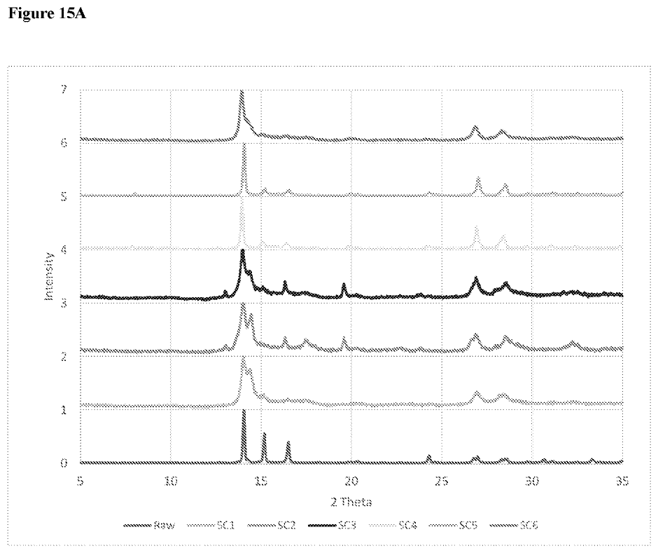

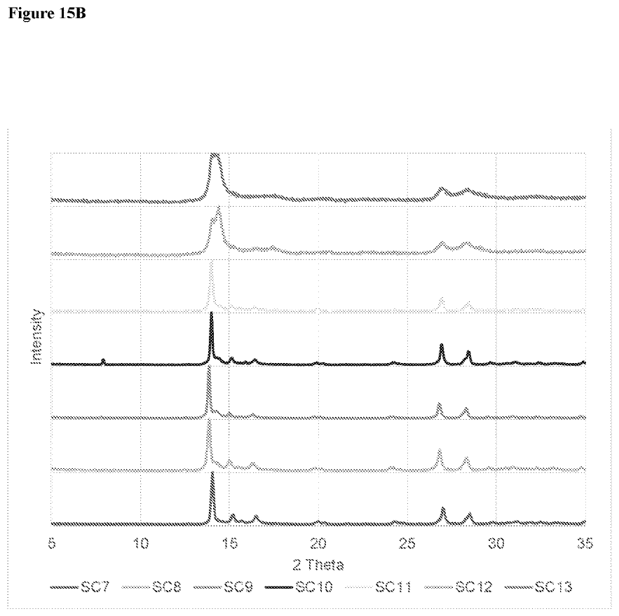

Powder X-ray diffraction analyses were conducted on a Siemens D5000 X-ray Diffractometer. PXRD scanned from 5 to 35 2θ degrees at a rate of 0.02 2θ degrees/second and 1 second per step. The raw material and all three SCP samples appeared to exhibit the same crystalline pattern, with the observable differences in intensity and broadening coming primarily from particle size effect and secondarily from preferred orientation. The diffraction pattern overlays are provided in FIG. 15.

### Specific Surface Area

Surface area analyses were conducted on a Quantachrome NOVAtouch™ LX2 BET Sorptometer. SC1 resulted in a 6.3x SSA increase when compared to the raw material and SC2 resulted in a 4.4x increase. Although SC3 did not yield sufficient material for analysis, based on PSD decrease, it is hypothesized that it also had a significant increase in SSA as well. Surface area results can be found in Table 1.

### Bulk Density

Bulk density analyses were conducted using a 10 mL graduated cylinder due to low sample volume. SC1 was the only precipitate measured due to insufficient materials available for analysis from SC2 and SC3. SC1 exhibited a bulk density decrease of ˜75% when compared to the raw material cisplatin. Bulk density results can be seen in Table 1.

### Conclusions

Cisplatin was successfully precipitated from all three solvent system tested, with DMF exhibiting the most promising results.

TABLE 1

| Run: | Raw | SC1 | SC2 | SC3 | SC4 | SC5 | SC6 | SC7 |
|---|---|---|---|---|---|---|---|---|
| Conditions | | Solvent | | | Pressure | | Temperature | |
| Solvent | | DMF | DMSO | 3:2 | DMF | DMF | DMF | DMF |
| | | | | DMSO:Acetone | | | | |
| Concentration | | 16.8 | 100.4 | 49.7 | 16.8 | 16.8 | 16.8 | 16.8 |
| (mg/mL) | | | | | | | | |
| Pressure | | X | X | X | High | Low | X | X |
| Temperature | | X | X | X | X | X | Low | High |
| scCO₂ Flowrate | | X | X | X | X | X | X | X |
| Sonication | | X | X | X | X | X | X | X |
| Feed Rate | | X | X | X | X | X | X | X |
| Yield | | 85.6% | 54.1% | 18.6% | 84.2% | 76.3% | 45.7% | 87.5% |
| PSD- | | | | | | | | |
| Master sizer | | | | | | | | |
| Dᵥ₁₀ (μm) | 1.72 | 0.668 | 0.777 | 0.646 | 0.793 | 0.729 | 0.709 | 0.762 |
| Dᵥ₅₀ (μm) | 10.9 | 1.89 | 11.80 | 1.87 | 5.30 | 2.29 | 1.84 | 3.11 |
| Dᵥ₉₀ (μm) | 25.9 | 9.18 | 26.6 | 4.18 | 14.5 | 10.6 | 6.83 | 10.1 |
| Surface Area | 2.44 | 15.46 | 10.83 | Insufficient | 3.98 | 4.97 | 10.31 | 4.39 |
| (m²/g) | | | | Material | | | | |
| Bulk Density | 1.01 | 0.25 | Insufficient | Insufficient | 0.496 | 0.707 | 0.346 | 1.65 |
| (g/mL) | | | Material | Material | | | | |
| | Run: | SC8 | SC9 | SC10 | SC11 | SC12 | SC13 | SC14 |
| | Conditions | scCO₂ Flow | | Sonication | | | Combination | |
| | Solvent | DMF | DMF | DMF | DMF | DMF | DMF | DMF |
| | Concentration | 16.8 | 16.8 | 16.8 | 16.8 | 16.8 | 16.8 | 16.8 |
| | (mg/mL) | | | | | | | |
| | Pressure | X | X | X | X | X | X | TBD |
| | Temperature | X | X | X | X | X | Low | TBD |
| | scCO₂ Flowrate | High | Low | X | X | X | X | TBD |
| | Sonication | X | X | High | Low | None | Low | TBD |
| | Feed Rate | X | X | X | X | X | X | TBD |
| | Yield | 56.84% | 73.44% | 82.71% | 95.18% | 49.38% | 51.72% | TBD |
| | PSD- | | | | | | | |
| | Master sizer | | | | | | | |
| | Dᵥ₁₀ (μm) | 0.785 | 0.526 | 0.582 | 0.745 | 0.706 | 0.738 | TBD |
| | Dᵥ₅₀ (μm) | 2.47 | 1.50 | 1.48 | 2.7 | 1.81 | 1.90 | TBD |
| | Dᵥ₉₀ (μm) | 11.7 | 3.56 | 3.76 | 16.9 | 4.49 | 6.46 | TBD |
| | Surface Area | 8.09 | 4.41 | 5.63 | 5.75 | 20.54 | 24.88 | TBD |
| | (m²/g) | | | | | | | |
| | Bulk Density | 0.599 | 0.346 | 0.384 | 1.74 | 0.223 | 0.374 | TBD |
| | (g/mL) | | | | | | | |

### MMAD determination

Approximately 100 mg (total, for 3 replicates) or each of 2 samples of cisplatin particles as described herein, SC9 with a lower specific surface area (4.41 m²/gm) and SC12 with a much higher surface area (20.54 m²/gm), were analyzed for MMAD on an APS 3321 spectrometer. The bulk density was 0.346 gm/cm³ for SC9 and 0.223 gm/cm³ for SC12. The results were as follows:
Low surface area sample: 1.73 um MMAD with a GSD (geometric standard deviation) of 1.44
Higher surface area sample: 1.71 um MMAD with a GSD of 1.64

The MMAD values were very close to the Dv50 values we obtained for the physical particle size distribution of the particles, 1.50 um and 1.81 um. This data demonstrates that the particles can be produced with MMADs permitting delivery by dry powder inhalation.

### SCP-Cisplatin Pilot Study

55 CR female NCr nu/nu mice 8-12 weeks of age at start date were injected subcutaneously in the flank with 1×10⁷ H69 tumor cells in 50% Matrigel; cell injection volume was 0.1 mL/mouse. A pair match was performed when tumors reach an average size of 100-150 mm³, after which treatment was initiated as detailed in Table 2.

TABLE 2
Drugs and Treatment:

| Gr. | N | Agent | Formulation dose | Route | Schedule |
|---|---|---|---|---|---|
| 1^{#} | 5 | No Treatment | — | — | — |
| 2 | 5 | vehicle | — | intra-tumoral | qd × 1 |
| 3 | 5 | Cisplatin | 25 μg/animal* | intra-tumoral | qd × 1 |
| 4 | 5 | SCP-Cisplatin | 25 μg/animal* | intra-tumoral | qd × 1 |
| 5 | 5 | SCP-Cisplatin | 125 μg/animal** | intra-tumoral | qd × 1 |

^{#}Control Group
*Approximately 1.13 mg/kg based on 25 uL intra-tumoral administration in a 22-gram mouse.
**Approximately 5.7 mg/kg based on 25 uL intra-tumoral (IT) administration in a 22-gram mouse.
Note:
Vehicle = a solution of 47.5% glycerin, 47.5% ethanol, and 5% water,
Cisplatin = cisplatin dissolved in saline solution,
SCP-Cisplatin = suspension of SCP processed cisplatin particles suspended in a solution of 47.5% glycerin, 47.5% ethanol, and 5% water.

Tumor cells implanted on Day 0 and treatments initiated on Day 18 (Mean TV=126 mm³). There were 5 treatment groups (n=5/group); all receiving IT injections =25 uL; 27G needle. The IT vehicle group was administered ethanol/glycerin/water. Animals were sacrificed at humane endpoint of TV=2000 mm³ or Study Day 51.

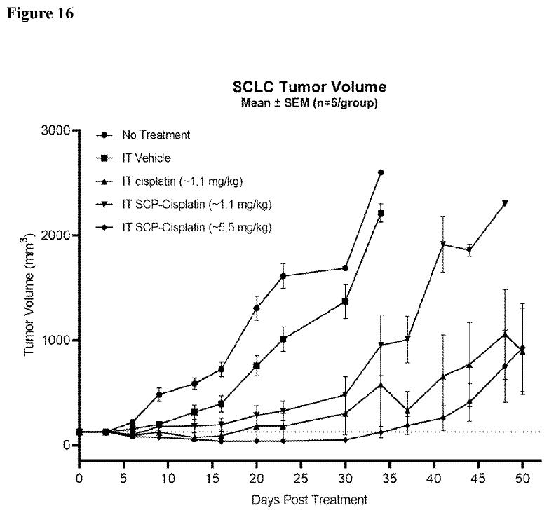

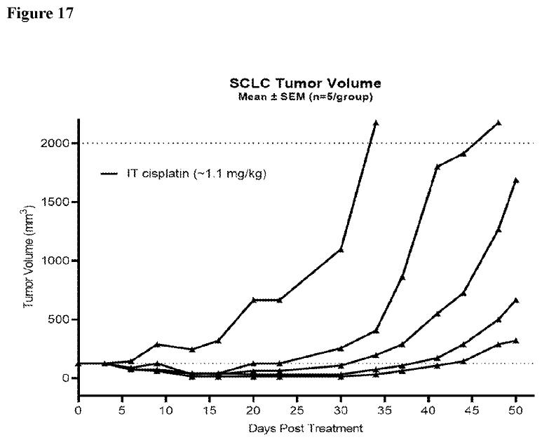

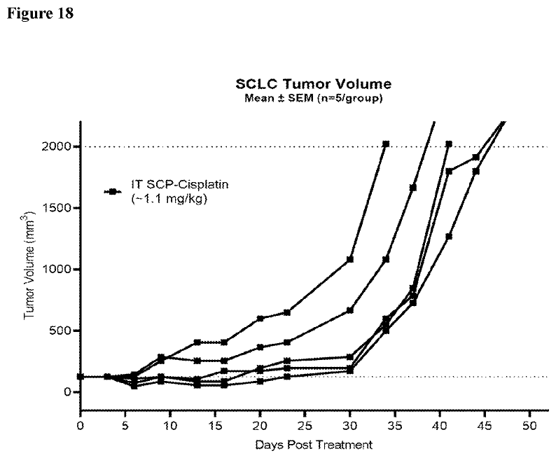

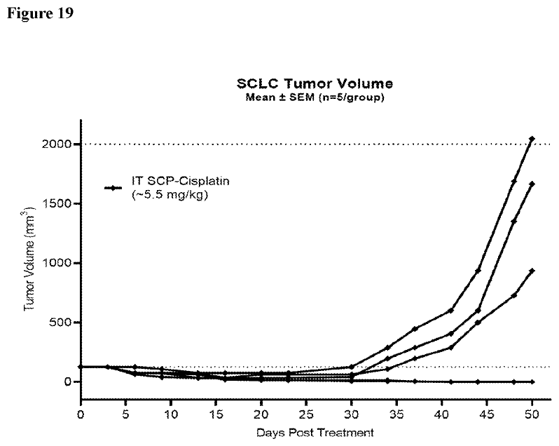

Data are shown in FIGS. 16-19. On Day 51 following a single IT injection:
3 of 5 animals in Group 3 survived, each with increasing tumor volume;
0 of 5 animals in Group 4 survived; and
5 of 5 animals in Group 5 survived. 3 of 5 had increasing tumor volume while the other 2 showed no measurable tumor (see FIG. 19).

We claim:

1. A composition, comprising particles comprising at least 95% by weight of cisplatin, wherein the particles have a specific surface area (SSA) of at least 3.5 m²/g.

2. The composition of claim 1, wherein the particles have a SSA of at least 4 m²/g.

3. The composition of claim 1, wherein the particles have a SSA of at least 10 m²/g.

4. The composition of claim 1, wherein the particles have a SSA of between 3.5 m²/g and about 30 m²/g.

5. The composition of claim 1, wherein the particles have a mean particle size by volume distribution (Dv50) of between about 1.0 micron to about 12 microns in diameter.

6. The composition of claim 1, wherein the particles have a mean bulk density between about 0.020 g/cm³ and about 0.8 g/cm³.

7. The composition of claim 1, wherein the particles comprise at least 98% by weight of cisplatin.

8. The composition of claim 1, wherein the particles are uncoated and exclude polymer, protein, polyethoxylated castor oil and polyethylene glycol glycerides composed of mono-, di- and triglycerides and mono- and diesters of polyethylene glycol.

9. The composition of claim 1, wherein the composition comprises a suspension further comprising a pharmaceutically acceptable liquid carrier.

10. The composition of claim 1, further comprising one or more components selected from the group consisting of polysorbate, methylcellulose, polyvinylpyrrolidone, mannitol, and hydroxypropyl methylcellulose.

11. The composition of claim 9, wherein the suspension is aerosolized, and the mass median aerodynamic diameter (MMAD) of aerosol droplets of the suspension may be any suitable diameter for use, such as between about 0.5 μm to about 6 μm diameter.

12. The composition of claim 1, wherein
(a) the composition is a dry powder composition, wherein the dry powder composition does not comprise a carrier or any excipients, and wherein the dry powder composition is aerosolized, and the MMAD of the aerosolized dry powder composition may be any suitable diameter for use, such as between about 0.5 μm to about 6 μm in diameter, or
(b) the composition is a dry powder composition, wherein the dry powder composition comprises a pharmaceutically acceptable dry powder carrier comprising one or more dry powder excipients, and wherein the dry powder composition is aerosolized, and the MMAD of the aerosolized dry powder composition may be any suitable diameter for use, such as between about 0.5 μm to about 6 μm in diameter.

13. A method for treating a tumor, comprising administering to a subject with a tumor an amount effective to treat the tumor of the composition of claim 1.

14. The method of 13, wherein
(a) the tumor is a carcinoma, a breast tumor, pancreatic tumor, prostate tumor, bladder tumor, lung tumor, ovarian tumor, gastrointestinal tumor, testicular tumor, cervical tumor, head and neck tumor, esophageal tumor, mesothelioma, brain tumor, neuroblastoma, or renal cell tumor, including but not limited to wherein the tumor is a metastatic testicular tumor, metastatic ovarian tumor, or advanced bladder cancer; and/or
(b) the method further comprises administering an additional therapeutic to the subject, including but not limited to anthracyclines, antimetabolites, alkylating agents, alkaloids, taxanes (including but not limited to paclitaxel, docetaxel, cabazitaxel, and combinations thereof), and/or topoisomerase inhibitors.

15. The method of claim 13, wherein the subject is a human subject.

16. The method of claim 13, wherein the composition is administered by intra-tumoral injection, peri-tumoral injection, intra-peritoneal injection, pulmonary administration, or is administered into a mammary fat pad.

17. A method for making compound particles, comprising:
(a) introducing (i) a solution comprising at least one solvent including but not limited to DMF (dimethylformamide), DMSO (dimethyl sulfoxide), acetone, or combinations thereof, or combinations thereof, and at least one solute comprising cisplatin into a nozzle inlet, and (ii) a compressed fluid into an inlet of a vessel defining a pressurizable chamber;
(b) passing the solution out of a nozzle orifice and into the pressurizable chamber to produce an output stream of atomized droplets, wherein the nozzle orifice is located between 2 mm and 20 mm from a sonic energy source located within the output stream, wherein the sonic energy source produces sonic energy with an amplitude between 10% and 100% during the passing, and wherein the nozzle orifice has a diameter of between 20 μm and 125 μm;
(c) contacting the atomized droplets with the compressed fluid, to cause depletion of the solvent from the atomized droplets, to produce cisplatin particles comprising at least 95% cisplatin, wherein the cisplatin particles have a specific surface area (SSA) of at 3.5 m²/g and have a mean particle size of between about 0.7 μm and about 8 μm,
wherein steps (a), (b), and (c) are carried out under supercritical temperature and pressure for the compressed fluid.

18. The method of claim 17, further comprising:
(d) contacting the compound particles produced in step (c) with an anti-solvent to cause further depletion of the solvent from the compound particles, wherein step (d) is carried out under supercritical temperature and pressure for the anti-solvent.
